(19) United States
(12) Patent Application Publication
(10) Pub. No.: US 2019/0070404 A1
(43) Pub. Date: Mar. 7, 2019

(54) LUER ACCESS DEVICE CAPPING AND CLEANSING DEVICES

(71) Applicant: CleanSite Medical, Inc., Solana Beach, CA (US)

(72) Inventor: Nicholas ANDERSON, Brooklyn, NY (US)

(73) Assignee: CleanSite Medical, Inc., Solana Beach, CA (US)

(21) Appl. No.: 16/059,029

(22) Filed: Aug. 8, 2018

Related U.S. Application Data
(60) Provisional application No. 62/542,770, filed on Aug. 8, 2017.

(51) Int. Cl. A61M 39/16; A61M 39/18; A61M 39/20

(52) U.S. Cl. CPC A61M 39/162; A61M 2205/0205; A61M 39/20; A61M 39/18

(57) ABSTRACT

Capping and cleansing devices for capping and cleansing medical connectors, particularly luer access devices, and methods for using such articles, are described. Such articles include a cap, a compressible cleansing matrix attached to the cap, and a resilient inner body disposed in the cap such that cap can mechanically engage and disengage the resilient inner body so as to allow the cap to rotate in unison with the resilient inner body when the two components are engaged or to rotate in relation to the resilient inner body when the two components are disengaged.

### RELATED APPLICATIONS

This application claims the benefit of and priority to U.S. provisional patent application Ser. No. 62/542,770, filed 8 Aug. 2017, and having the same title as this application, which application is hereby incorporated by reference in its entirety for any and all purposes.

### TECHNICAL FIELD OF THE INVENTION

This invention is directed to cleansing devices for cleansing and capping medical devices, particularly luer access devices such as needleless, valved connectors, and methods for using such articles.

### BACKGROUND OF THE INVENTION

### 1. Introduction

The following description includes information that may be useful in understanding the present invention. It is not an admission that any such information is prior art, or relevant, to the presently claimed inventions, or that any publication specifically or implicitly referenced is prior art.

### 2. Background

In the medical field, and in particular the area of infusion of fluids or aspiration of fluids to or from a patient, there remains a need to prevent the transmission of pathogens into or onto a patient from a potentially contaminated surface of a medical device such as a luer access device, for example, a needleless, valved connector. Pathogens include microorganisms such as bacteria and viruses, the transmission of which into a patient may result in an infection that could be life threatening. Specific to healthcare settings, the term “nosocomial infection” describes those infections that originate from or occur in a hospital or hospital-like setting. In the U.S., nosocomial infections are estimated to occur in at least 5% of all acute care hospitalizations. The estimated incidence is more than two million cases per year, resulting in significant morbidity, mortality, and an expense. Indeed, nosocomial infections are estimated to more than double the mortality and morbidity risks of any admitted patient, and likely result in about 90,000 deaths a year in the United States. Common sites for such transmissions are found on such medical devices as luer access devices, vials, needleless (or needle free) valves, and the injection ports of vessels, tubing, and catheters. The incidence of such infections in patients is increasing, and infection control practitioners (ICP's) often cite improper cleansing of sites as a major source of these infections.

As described above, exposure to pathogens and infectious agents (e.g., pathogenic bacteria, viruses, fungi, etc.) in medical settings is a matter of serious concern. One route of exposure to such agents is the opening made in skin provided by the bore of needle, cannula, or other similar device used to provide access to a patient's vasculature. It is known that patients whose skin has been compromised in this way are at increased risk for developing serious blood stream infections. In the United States alone, approximately 300,000 blood stream infections per year result from the installation and use of peripheral intravenous catheters (PIVC), and more than 80,000 blood stream infections are associated with the use central venous catheters (CVC). All told, in the U.S. approximately 20,000 patients die annually from hospital-acquired infections that result from PIVC and CVC use. Costs associated with the care and treatment of patients that develop infections due to PIVC and CVC use exceed $2.7 billion annually.

In hospital settings today, occupational health and safety regulations designed reduce the risk to health care workers from needle prick and similar injuries have resulted in the deployment of needleless medical valves whenever possible. Currently, more than 500 million needleless valves are used annually in hospitals throughout the U.S. Needleless valves are used primarily in conjunction with PIVC and CVC devices, which may contain from as few as one to as many as 3, 4, 5, or more needleless valves.

FIG. 1 illustrates an example of a representative medical valve in use today. The widespread use of needleless valves in acute medicine has contributed to a marked increase in the incidence of hospital-acquired infections (HAIs), particularly blood stream infections. To reduce the risk of infection from a contaminated needleless valve, standard practice today requires that a nurse or other health care worker clean the surface of a needleless valve by rubbing it with a sterile alcohol swab or wipe immediately prior to making a connection to the valve, for example, attaching a syringe to the valve to deliver a medication via a PIVC already connected to a patient. Given the magnitude of the mortality and morbidity associated with HAIs and the large number of blood stream infections that result from PIVC and CVC use, a long-recognized yet significant unmet need exists for articles or devices that can be used to reduce or eliminate the risk of initiating an HAI merely by accessing a patient's vasculature through a needleless valve component of a PIVC or CVC inserted into a blood vessel of a patient.

Traditionally, cleaning or cleansing a potentially contaminated surface involved a protocol of alcohol swabbing prior to making the necessary connections to the site. Alcohol swabs are typically a small pad of cotton gauze soaked in isopropyl alcohol, packed individually in a foil package to prevent evaporation of the isopropyl alcohol from the swab prior to use. Properly used, the package is opened at or near the site to be swabbed. With gloved hands, the swab is removed by a nurse or other healthcare provider and wiped across the top and side surfaces of the sites to be connected. After use, the swab and foil package are discarded and the site allowed to dry, usually 20-30 seconds, immediately prior to making any connection. This “drying” period is important because, as the alcohol dries, it breaks open the cellular walls of microorganisms, thereby killing them.

Unfortunately, because of increased duties and responsibilities, shrinking nursing staffs, and inadequate training, alcohol swabbing is often not performed or is poorly executed. A poorly swabbed site can carry microorganisms that, if allowed to enter a patient's body, can cause serious infection. In addition, supervisory oversight is nearly impossible, because unless a supervisor actually observes swabbing as it is performed, the supervisor cannot know whether or not it was done properly or performed at all. Further, without at least a sufficient microscopic examination for microbial residue (e.g., biofilm), there may be no evidence of an alcohol swab being performed.

Thus, a significant need still exists for devices and techniques cleanse sites on medical devices prior to their use with or connection to patients, and which eliminate technique-related and training issues and provide an unequivocal indicator that a site is clean prior to accessing a patient's vascular system.

### 3. Definitions

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

As used herein, the singular forms “a”, “an”, and “the” include plural references unless the context clearly dictates otherwise.

As used herein, the term “about” refers to approximately a +/−10% variation from the stated value. It is to be understood that such a variation is always included in any given value provided herein, whether or not it is specifically referred to.

A “patentable” composition, process, machine, or article of manufacture according to the invention means that the subject matter at issue satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically excludes the unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application is filed or issues as a patent to a time the validity of one or more of the appended claims is questioned, the claims are to be interpreted in a way that (1) preserves their validity and (2) provides the broadest reasonable interpretation under the circumstances.

A “plurality” means more than one.

The term “species”, when used in the context of describing a particular compound or molecule species, refers to a population of chemically indistinct molecules.

### SUMMARY OF THE INVENTION

The object of the invention is to address these long-standing but still unmet needs. This invention addresses these needs by providing patentable, single-use cleansing and devices or articles that can be used to effectively and efficiently cap and cleanse, and preferably sterilize, exposed surfaces of medical articles such as luer access devices, e.g., needleless medical valves, particularly the accessible surface(s) of the valve stems of needleless valves, particularly those surfaces that may become contaminated with pathogens or infectious agents and form part of the fluid communication pathway between an external fluid source and a patient's blood stream. In the context of the invention, “cleanse” encompasses cleaning, disinfecting, sanitizing, and/or sterilizing, whereas “capping” refers to using a device, i.e., a “cap”, to cover a surface, or set of surfaces, of a medical connector such as a needleless valve, so as to limit or prevent exposure of such surface(s) to the environment (e.g., the air circulating in a hospital's intensive care unit) for a period longer than necessary to cleanse the desired surface(s) of a medical connector.

Thus, in one aspect, the invention provides capping and cleansing devices for medical devices such as luer access devices, including needless, valved connectors. In general, such capping and cleansing devices include a cap, a resilient inner body associated with the cap such that the cap can engage and disengage from the resilient inner body so as to allow the cap to rotate in relation to the resilient inner body, and a compressible cleansing matrix disposed in the cap and positioned to contact one or more exterior surface(s) of a medical connector connected to the capping and cleansing device of the invention.

The resilient inner body of a capping and cleansing device of the invention has a wall that bounds a central, interior (preferably cylindrical) bore that spans between oppositely disposed first and second (or upper and lower, respectively) openings. One or more thread-engaging tabs (or threads), preferably two (or more) thread-engaging tabs, are oppositely disposed on the interior wall of the bore, preferably near the lower opening. The thread-engaging tab(s) (or threads) is(are) are configured to engage a complementary threaded region on the exterior surface of, for example, a needleless medical valve such the capping and cleansing device can be securely threaded onto the targeted threaded portion of a medical connector for capping and, if desired, cleansing.

In preferred embodiments, the exterior surface of the resilient inner body includes a cap-engaging region that includes one or more structures that allow the resilient inner body to mechanically engage and disengage the cap. Preferred embodiments of cap-engaging structures include alternating teeth and grooves arrayed about the outer circumference of the resilient inner body, which teeth and grooves are complementary to one or more spaced engaging structures (e.g., teeth) disposed on the inner surface of the cap wall. In other embodiments, the cap-engaging region of the resilient inner body is disposed on its inner surface to engage one or more complementary engaging structures (e.g., teeth) arrayed on the exterior or outer surface of matric well wall.

The resilient inner body also includes a compressible region located above the thread-engaging tab(s)(or threads) and cap-engaging region. The compressible region is any structure that allows the resilient inner body to be compressed so as to bring the first and second openings closer together. In preferred embodiments, the compressible region is a torsion spring, which in certain particularly preferred embodiments is a molded torsion spring formed, preferably during injection molding, as part of the resilient inner body. Compression of the compressible region is achieved by applying a downward force to the cap, a portion of the top inner surface of which bears on the upper surface of the resilient inner body's compressible region. Such compression causes the locking components of the cap and resilient inner body to disengage, which allows a user to rotate the cap in relation to the resilient inner body when it is securely yet releasably connected to a medical connector, for example, via complementary threads on the resilient inner body and threaded region of a medical connector.

As those in the art will appreciate, in some embodiments, the resilient inner body may be formed as two or more parts that are assembled, one on top of the other, during assembly of the capping and cleansing device of the invention. For example, in some embodiments where the resilient inner body is made of two parts, the upper part comprises the compressible region (e.g., a torsion spring formed during injection molding of the part), while the lower part comprises the cap-engaging region and the thread-engaging tab(s)(or threads). In contrast, a representative three-part embodiment of a resilient inner body includes an upper section that comprises the first (upper) opening and the compressible region, a midsection that comprises the cap-engaging region, and a lower section that includes the thread-engaging tab(s) (or threads) and second (lower) opening. In embodiments of the invention where the resilient inner body is made from two or more parts, those parts, once assembled, preferably are mechanically connected such that they can move in unison, for example, when the capping and cleansing device of which they are a part is threaded onto the threaded portion of a medical connector, for example, a needleless medical valve. Any suitable mechanical lock, and corresponding set of mechanical structures, can be used to link such parts together.

In some preferred embodiments, the resilient inner body also includes a sealing member configured to provide a fluid tight seal between the capping and cleansing device of the invention and a medical connector connected thereto. The seal will preferably be disposed in a channel formed in the inner surface of the wall of the resilient inner body proximate to the second (lower) opening, typically below the thread-engaging tab(s)(or threads). In some of these embodiments, the cap may include one or more vents to allow fluid and/or air from inside the device to escape as the capping and cleansing device is secured to a medical connector, while in other embodiments, no vent(s) is(are) provided.

Devices of the invention also include a cap. The cap includes an outer cavity formed by a curved outer wall that is joined to a top portion about its periphery and a preferably concentric central matrix well that extends from the top into the outer cavity. The wall forming the matrix well is spaced from the cap's outer wall to form a resilient member housing that can be accessed through an opening created by the gap between the wall of the outer cavity and the matrix well wall. In preferred embodiments, the inner surface of the outer wall includes one or more locking structures (e.g., teeth) designed to releasably engage complementary structures in the cap-engaging region of the resilient inner body. In other embodiments, the cap's locking structure(s) is (are) disposed on the outer surface of the matrix well wall, which locking structure(s) is (are) designed to releasably engage complementary structures in the cap-engaging region on the inner wall of the resilient inner body. Engagement of cap's locking structure(s) with those in the cap-engaging region of the resilient inner body allow a user to rotate the cap and resilient inner body in unison, for example, as a capping and cleansing cap is screwed onto the threaded portion of a medical connector to be cleansed and/or capped. Once the device is releasably secured to a medical connector, the cap's locking structure(s) can be disengaged from those in the cap-engaging region of the resilient inner body, thereby allowing a user to rotate the cap about its central axis in relation to the resilient inner body.

The cap and its various components are preferably formed as a single, integral unit during manufacturing (e.g., by injection molding).

Devices of the invention also include a compressible cleansing matrix disposed in the cap's matrix well. The compressible cleansing matrix, for example, an open-cell foam, is preferably secured to an inner surface of the cap so as to limit or restrict rotation of the cleansing matrix during cap rotation. The compressible cleansing matrix is configured to contact and cleanse one or more surfaces of a medical connector that contacts the matrix upon a medical connector's association with a capping and cleansing device of the invention. The compressible cleansing matrix attached to the cap can be axially compressed (i.e., compressed along the central axis of the cap's matrix well) upon insertion of a medical connector into such a capping and cleansing device. Because the medical connector surface(s) to be cleansed may be contaminated with microorganisms that form a biofilm (i.e., a matrix of microorganisms and extracellular material attached to a surface, which enables the microorganisms, typically bacteria and/or fungi, to adhere to a surface and carry out certain biochemical processes), the compressible cleansing matrix also preferably has sufficient mechanical integrity when compressed to allow its use to disrupt any biofilm that may be present on the surface of the medical connector, as can occur by rotating, twisting, or otherwise moving the then-compressed cleansing matrix in relation to the medical connector (e.g., a needleless medical valve), for example, by rotating the cap (to which the compressible cleansing matrix is attached) in relation to the resilient inner body of the capping and cleansing device and the medical connector to which resilient inner body is releasably attached. The resulting friction between the compressed cleansing matrix and surface of the medical connector disrupts the biofilm, thereby cleansing, and preferably sterilizing, the medical connector. Leaving the capping and cleansing device secured to (i.e., capping) the medical connector after such cleansing will limit, and preferably preclude, biofilm regrowth and/or the microbial recolonization of cleansed surfaces (which remain in contact with the compressible cleansing matrix).

In preferred embodiments, the compressible cleansing matrix includes one or more cleansing agent species dispersed therein, preferably at the time the article is manufactured, although in some embodiments, the cleansing agent may be dispersed into the matrix just prior to the matrix coming into contact with a medical connector. In embodiments of the latter sort, the cleansing agent is preferably housed in the body of the capping and cleansing device in a reservoir configured to rupture upon association of a medical connector for cleansing. Such a reservoir can be disposed between the matrix and medical connector, or, more preferably, between the rotatable cap and compressible cleansing matrix. Preferred cleansing agents include antimicrobial agents such as isopropyl alcohol, chlorhexidine, and silver ions. In some embodiments, the capping and cleansing device of the invention will include a valve or opening to allow liquid in the cleansing agent to evaporate.

In some preferred embodiments, the compressible cleansing matrix includes two or more portions. In some of such embodiments, one portion of the matrix is attached to the inner surface of the cap and another portion is secured to the inner surface of the wall forming the matrix well. If present, the portion of the compressible cleansing matrix secured to the inner surface of the matrix well wall is preferably configured to radially compress upon association with a medical connector to be capped and cleansed.

In preferred embodiments, the capping and cleansing devices include a removable seal attached to the cap to seal the device, thus separating the interior spaces and structures of the cap from the external environment. Such a seal prevents exposure of the cap's interior, the resilient inner body, and compressible cleansing matrix to the environment until the seal is removed, typically by a healthcare worker just prior to her/his use of the capping and cleansing device to cap and then, if desired, to clean/cleanse the medical connector (e.g., needleless medical valve) to which it is connected. In preferred embodiments, such cleansing substantially disrupts any biofilm that may exist on surfaces contacted by the compressible cleansing matrix. If desired, the capping and cleansing device can be left in place (typically after cleansing the medical connector attached thereto) in order to cap the medical connector until it is further accessed, thereby minimizing exposure of capped exterior surfaces of the connector to potential pathogen contamination (and biofilm formation) from the surrounding environment. Seals are typically installed during manufacture of a capping and cleansing device of the invention. In those embodiments where the capping and cleansing devices are sterilized during manufacture (e.g., by irradiation, exposure to ethylene oxide, etc.), seals are preferably applied prior to sterilization.

In various embodiments, the outer surface of the cap of a capping and cleansing device according to the invention includes one or more grip-enhancing structures or coatings, e.g., a plurality of vertical ridges. Such grip-enhancing structures or coatings facilitate a user's grasp of the body of a capping and cleansing device between her/his fingers, which can be helpful not only during insertion and removal of a medical connector from the capping cleansing device, but also during the cleansing process, where the user rotates the cap in relation to the resilient inner body in order to scrub and thereby clean/cleanse the surface(s) of the inserted medical connector with the compressible cleansing matrix.

Other aspects of the invention concern methods of cleansing and/or capping medical connectors using a capping and cleansing device according to the invention. Such methods typically involve compressing the resilient inner body of such a device after it has been connected to a medical connector as to allow the cap to spin or rotate in relation to the resilient inner body. Such compression facilitates contact between the device's compressible cleansing matrix and the associated surface(s) of the medical connector. Spinning or rotation of the cap in relation to the resilient inner body, and the associated surface(s) of the medical connector, allow those surfaces to be scrubbed, thereby cleansing them. Preferably, such cleansing methods provide for the disruption of any biofilm present on the surface(s) of the medical connector associated the capping and cleansing device. And in those embodiments where the compressible cleansing matrix contains one or more antimicrobial agents, microbes and pathogens present in such biofilm and/on on such surface(s) are destroyed or rendered nonviable.

Features and advantages of the invention will be apparent from the following detailed description, and appended claims.

### BRIEF DESCRIPTION OF THE DRAWINGS

These and other aspects will now be described in detail with reference to the following drawings. Unless otherwise indicated, it is understood that the drawings are not to scale, as they are intended merely to facilitate understanding of the invention as opposed to specific dimensions, etc. In the drawings, like numbers in two or more drawings represent like elements.

FIG. 1 shows several drawings ((a)-(g)) of a representative capping and cleansing device of the invention, its constituent parts (views (b)-(g)), and the device associated with a medical connector (view (a)).

FIG. 2 shows an exploded view (a) of a representative capping and cleansing device of the invention and a medical connector and several cross-sectional views ((b)-(d)) of a sealed representative capping and cleansing device of the invention (view (b)) and such a capping and cleansing device capping a medical connector (views (c) and (d)).

FIG. 3 shows six different views of a representative capping and cleansing device of the invention. Views (a)-(c) show the device in a static position, where the cap and resilient inner body are engaged such that the cap, and hence the compressible cleansing matrix associated therewith, cannot rotate in relation to the device's resilient inner body. Views (d)-(f) show the same representative device with the cap and resilient inner body in movable relation such that the cap (and the compressible cleansing matrix associated therewith) can be rotated in relation to the device's resilient inner body.

FIG. 4 shows views of the cap portion of a representative capping and cleansing device of the invention. View (a) shows a top view of the cap portion. View (b) shows a side view of the cap portion. View (c) shows a bottom view of the cap portion. View (d) shows a cross-sectional view of the cap portion. Representative measurements of this particular embodiment are shown on views (b) and (d).

FIG. 5 shows seven different views ((a)-(g)) of the resilient inner body portion of a representative capping and cleansing device of the invention. Representative measurements of this particular embodiment are shown on several of the views.

FIG. 6 shows five different views ((a)-(e)), three of which show a compressible cleansing matrix portion of a representative capping and cleansing device of the invention. Views (a)-(c) show top, side, and bottom views of this particular compressible cleansing matrix. Views (d) and (e) show bottom and side views of a seal portion of a representative capping and cleansing device of the invention.

The details of one or more embodiments are set forth in the accompanying drawings and the description below. Other features and advantages will be apparent from the description and drawings, and from the claims.

### DETAILED DESCRIPTION

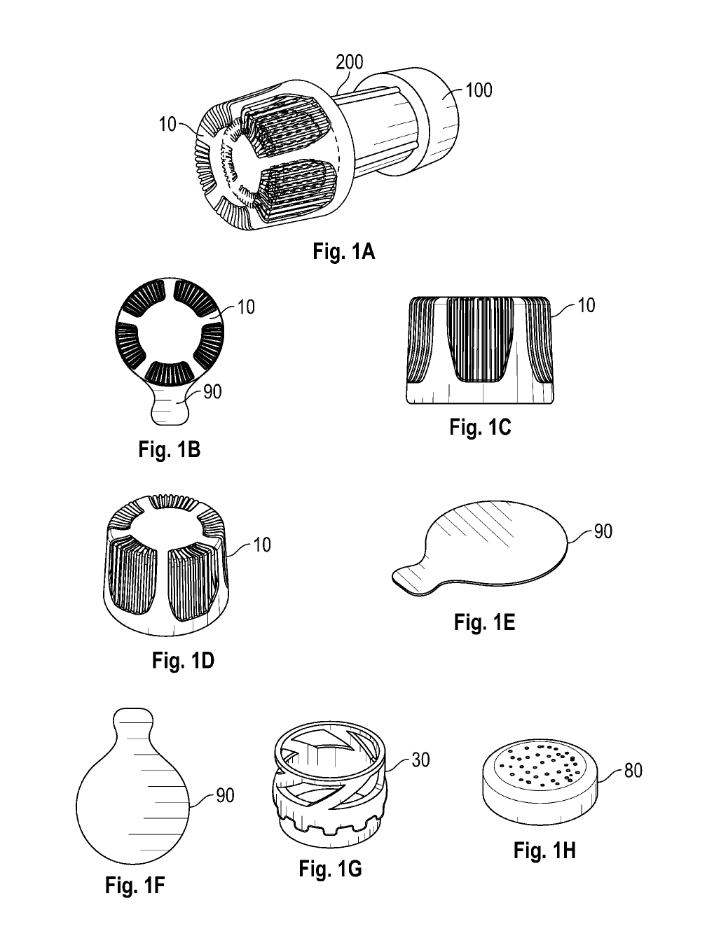

In the following detailed description, reference is made to the accompanying figures (FIGS. 1-6), which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the spirit or scope of the subject matter presented here.

This invention concerns patentable single-use capping and cleansing devices that can be used to effectively and efficiently clean, disinfect, and preferably sterilize, exposed surfaces of medical connectors, particularly those of luer access devices such as needleless medical valves that at times become part of a fluid communication pathway for introduction of fluids (e.g., IV fluids, blood, plasma, medicines, etc.) into a patient, as these surfaces are at risk for contamination with pathogens and infectious agents such as bacteria, fungi, and viruses. “Single-use” (or “single purpose”) refers to an article or device suitable for one use or purpose only, as distinguished from “dual” or “multiple” use or purpose devices. Thus, in the context of the invention, a “single-use” capping and cleansing device is one that is useful for cleansing, for example, a needleless medical valve. After the cleansing operation, the device may, if desired, be left in place on the medical connector in order to prevent recontamination of its cleansed surface(s) after the capping and cleansing device of the invention is removed. After removal, the device is preferably discarded. Prior to removal, however, the capping and cleansing device can again be used to cleanse the capped surface(s) of the medical connector.

In general, a capping and cleansing device of the invention comprises a resilient inner body disposed within a cap that can be rotated or turned in relation thereto when the capping and cleansing device is secured to a medical connector, and a compressible matrix element containing one or more antimicrobial agents and having a structure to allow capped surfaces of the medical connector to be cleansed. More specifically, the resilient inner body has a wall that forms a central, interior (preferably cylindrical) bore that extends between oppositely disposed first and second (or upper and lower, respectively) openings. The interior wall of the central bore nearer the second (lower) opening includes one or more thread-engaging tabs (or threads), preferably two thread-engaging tabs disposed opposite to each other. The thread-engaging tab(s) (or threads) is (are) are configured to engage a complementary threaded region on the exterior surface of, for example, a needleless medical valve. This allows the capping and cleansing device to be securely threaded onto the targeted threaded portion of a medical connector for cleansing and, if desired, capping that portion of the medical connector. The exterior surface of the resilient inner body includes one or more structures that allow it to mechanically engage and disengage complementary structures disposed on an inner surface of the cap.

In many embodiments, the resilient inner body's exterior surface includes a cap-engaging region that includes one or more structures such as spaced protrusions (e.g., teeth) that allow the resilient inner body to mechanically engage and disengage complementary engaging structures on an interior surface of the cap. Preferred embodiments of cap-engaging structures include alternating teeth and grooves (or channels) arrayed about the outer circumference of the resilient inner body, which teeth and grooves are complementary to one or more spaced engaging structures (e.g., teeth) disposed on the inner surface of the cap's outer wall. In other embodiments, the cap-engaging region of the resilient inner body is disposed on its inner surface for engagement with one or more complementary engaging structures (e.g., teeth) arrayed on the exterior or outer surface of matrix well wall. As will be appreciated, in configurations that include teeth and channels, the “teeth” can be raised protrusions and the “channels” can be the spaces or gaps between the raised protrusions.

The resilient inner body also includes a compressible region. In many preferred embodiments, it is located above the thread-engaging tab(s)(or threads) and cap-engaging region. The compressible region can be any structure that allows the resilient inner body to be compressed so as to bring the first and second openings closer together to allow disengagement of the engaging structures of the cap and resilient inner body. In preferred embodiments, the compressible region is a torsion spring, which in certain particularly preferred embodiments is a molded torsion spring formed from a plastic or other sufficiently flexible or resilient material, preferably during injection molding, as part of the resilient inner body.

In some embodiments, the resilient inner body is made from two or parts that are then assembled to form the complete inner body. For example, a resilient inner body can be formed as two or more separate parts that are assembled, one on top of the other, during manufacture of the capping and cleansing device of the invention. For instance, in embodiments where the resilient inner body is made of two parts, the upper part preferably comprises the compressible region (e.g., a torsion spring formed during injection molding of the upper part), while the lower part comprises the cap-engaging region and the thread-engaging tab(s) (or threads). In contrast, a representative three-part embodiment of a resilient inner body includes an upper section that comprises the first (upper) opening and the compressible region, a midsection that comprises the cap-engaging region, and a lower section that includes the thread-engaging tab(s) (or threads) and second (lower) opening. In an alternative three-part embodiment, the upper section comprises the first (upper) opening and cap-engaging region, the midsection comprises the compressible region, and the lower section includes the thread-engaging tab(s)(or threads) and second (lower) opening. As will be appreciated, the invention encompasses all possible combinations of parts having a cap-engaging region, compressible region, and a region to engage the threads of the threaded portion of a medical connector, with the proviso that the final combination be capable of being compressed to provide cap-engaging and -disengaging functionality such that when corresponding structures on the cap and inner body are engaged, the cap and inner body can rotate together about their central axes, and when the corresponding structures on the cap and inner body are disengaged by compression of the compressible region, a user can rotate the cap about its central axis in relation to, or independently from (i.e., the cap spins while the resilient inner body does not), the resilient inner body.

In embodiments of the invention where the resilient inner body is made from two or more parts, those parts, once assembled, preferably are mechanically connected such that they, too, move in unison, for example, when the capping and cleansing device of which they are a part is threaded onto the threaded portion of a medical connector, for example, a needleless medical valve. Any suitable mechanical lock, and corresponding set of mechanical structures, can be used to link such parts together.

In some preferred embodiments, the resilient inner body also includes a sealing member (i.e., seal) configured to provide a fluid tight seal between the capping and cleansing device of the invention and a medical connector connected thereto. The seal is preferably disposed in a channel formed in the inner surface of the wall of the resilient inner body proximate to the second (lower) opening, typically below the thread-engaging tab(s) (or threads).

Each device of the invention also includes a cap in operable association with a resilient inner body. A cap typically includes an outer cavity formed by a curved outer wall that is joined to a top portion, preferably about the top portion's periphery, and a preferably concentric central matrix well that extends from the top's inner surface into the outer cavity. The wall forming the matrix well is spaced from the cap's outer wall to form a resilient member housing that can be accessed through an opening created by the gap between the cap's wall and the matrix well wall. In many preferred embodiments, the inner surface of the cap's outer wall includes one or more engaging or locking structures (e.g., teeth) designed to releasably engage (i.e., corresponding structures can be engaged and disengaged, as desired) complementary structures in the cap-engaging region of the resilient inner body. In other embodiments, the cap's engaging or locking structure(s) is (are) disposed on the outer surface of the matrix well wall, which locking structure(s) is (are) designed to releasably engage complementary structures in the cap-engaging region on the inner wall of the resilient inner body. Engagement of cap's engaging or locking structure(s) with those in the cap-engaging region of the resilient inner body allows a user to rotate the cap and resilient inner body in unison, for example, as a capping and cleansing cap is screwed onto the threaded portion of a medical connector to be cleansed and/or capped. Once the device is releasably secured to a medical connector, the cap's locking structure(s) can be disengaged from those in the cap-engaging region of the resilient inner body, thereby allowing a user to rotate the cap about its central axis in relation to the resilient inner body. In some embodiments, the cap may include one or more vents to allow fluid and/or air from inside the device to escape as the capping and cleansing device is secured to a medical connector, while in other embodiments, no vent(s) is(are) provided.

The capping and cleansing devices of the invention also include a compressible cleansing matrix in the cap's matrix well. The compressible cleansing matrix can be, for example, an open-cell foam. The cleansing matrix is preferably secured to an inner surface of the cap so as to limit or restrict its rotation independent of the cap during cap rotation. The compressible cleansing matrix is configured to contact and cleanse one or more surfaces of a medical connector that contacts the matrix upon a medical connector's association with a capping and cleansing device of the invention. The compressible cleansing matrix attached to the cap can be axially compressed (i.e., compressed along the central axis of the cap's matrix well) upon insertion of a medical connector into a capping and cleansing device.

Because the medical connector surface(s) to be cleansed may be contaminated with microorganisms that form a biofilm (i.e., a matrix of microorganisms and extracellular material attached to a surface, which enables the microorganisms, typically bacteria and/or fungi, to adhere to a surface and carry out certain biochemical processes), the compressible cleansing matrix preferably has sufficient mechanical integrity when compressed to allow its use to disrupt any biofilm that may be present on a surface of the medical connector that is contacted by the cleansing matrix. Disruption of biofilm can occur by rotating, twisting, or otherwise moving a then-compressed cleansing matrix in relation to the medical connector (e.g., a needleless medical valve), for example, by rotating the cap (to which the compressible cleansing matrix is attached) in relation to the resilient inner body of the capping and cleansing device and the medical connector to which resilient inner body is releasably attached. The resulting friction between the compressed cleansing matrix and surface of the medical connector disrupts the biofilm, thereby cleansing, and preferably sterilizing, the medical connector. Leaving the capping and cleansing device secured to (i.e., capping) the medical connector after such cleansing will limit, and preferably preclude, biofilm regrowth and/or the microbial recolonization of cleansed surfaces (which remain in contact with the compressible cleansing matrix) of the medical connector.

In preferred embodiments, the compressible cleansing matrix includes one or more cleansing agent species dispersed therein, preferably at the time the article is manufactured, although in some embodiments, the cleansing agent may be dispersed into the matrix just prior to the matrix coming into contact with a medical connector. In embodiments of the latter sort, the cleansing agent is preferably housed in the body of the capping and cleansing device in a reservoir configured to rupture upon association of a medical connector for cleansing. Such a reservoir can be disposed between the matrix and medical connector, or, more preferably, between the rotatable cap and compressible cleansing matrix. Preferred cleansing agents include antimicrobial agents such as isopropyl alcohol, chlorhexidine, and silver ions. In some embodiments, the capping and cleansing device of the invention will include a valve or opening to allow liquid in the cleansing agent to evaporate.

In some preferred embodiments, the compressible cleansing matrix is comprised of two or more components. In some of such embodiments, one portion of the matrix is attached to the inner surface of the cap and another portion is secured to the inner surface of the wall forming the matrix well. If present, the portion of the compressible cleansing matrix secured to the inner surface of the matrix well wall is preferably configured to radially compress upon association with a medical connector to be capped and cleansed. When a cleansing matrix is comprised of two or more components, the matrix components may be made from the same of different material(s).

As described, the central matrix well is adapted to receive the compressible cleansing matrix. The surface(s) of the central matrix well in contact with the matrix preferably includes one or more retaining structures to retain the compressible cleansing matrix so as to link its rotation or movement to that of the cap, particularly when the engaging structures of the cap and resilient inner body are disengaged so as to allow cap rotation during a medical connector cleansing procedure. Such retaining structures include ridges and other protrusions from the surface of central matrix well in contact with the compressible cleansing matrix. An adhesive can also be used to adhere that portion of the compressible cleansing matrix to a desired position in the matrix well.

In various embodiments, the outer surface of the cap of a capping and cleansing device according to the invention includes one or more grip-enhancing structures or coatings, e.g., a plurality of vertical ridges. Such grip-enhancing structures or coatings facilitate a user's grasp of the body of a capping and cleansing device between her/his fingers, which can be helpful not only during insertion and removal of a medical connector from the capping cleansing device, but also during the cleansing process, where the user rotates the cap in relation to the resilient inner body in order to scrub and thereby clean/cleanse the surface(s) of the inserted medical connector with the compressible cleansing matrix.

The cap and resilient inner body can be made from any suitable material or combinations of different materials. Plastics are particularly preferred. The material(s) used to manufacture the cap may be the same or different as the material(s) used to produce the resilient inner body.

The cap and its various components are preferably formed as a single, integral unit during manufacturing (e.g., by injection molding). The cap and resilient inner body can be manufactured by any suitable process, including extrusion, injection molding, and additive manufacturing (e.g., 3D printing). After manufacturing, a resilient inner body is inserted into a cap to form a capping and cleansing device of the invention. For securing a cap to a resilient inner body, any suitable retaining structure, or group of structures, that provides for movement, i.e., rotation, of the cap in relation to the resilient inner body can be used. Such structures include attachment mechanisms such as “snap-fit” mechanisms where interacting parts are sufficiently flexible and have preferably have tapered surfaces so facilitate assembly.

A compressible cleansing matrix can be positioned in the matrix well before of after a resilient inner body and cap are operably associated. In preferred embodiments, a suitable adhesive is used to securely adhere the compressible cleansing matrix, or, if the matrix comprises two or more parts, its various portions, to one or more inner surfaces of the cap's matrix well. In some embodiments, the surface of the matrix well that contacts the compressible matrix includes a structure to assist in securely retaining the matrix in the well, thus ensuring that it moves in conjunction with the cap when the cap is rotated during a cleansing procedure.

In preferred embodiments, the capping and cleansing devices include a removable seal attached to the cap to seal the device, thus separating the interior spaces and structures of the cap from the external environment. Such a seal prevents exposure of the cap's interior, the resilient inner body, and compressible cleansing matrix to the environment until the seal is removed, typically by a healthcare worker just prior to her/his use of the capping and cleansing device to cap and then, if desired, to clean/cleanse the medical connector (e.g., needleless medical valve) to which it is connected. In preferred embodiments, such cleansing substantially disrupts any biofilm that may exist on surfaces contacted by the compressible cleansing matrix. If desired, the capping and cleansing device can be left in place (typically after cleansing the medical connector attached thereto) in order to cap the medical connector until it is further accessed, thereby minimizing exposure of capped exterior surfaces of the connector to potential pathogen contamination (and biofilm formation) from the surrounding environment. Seals are typically installed during manufacture of a capping and cleansing device of the invention. In those embodiments where the capping and cleansing devices are sterilized during manufacture (e.g., by irradiation, exposure to ethylene oxide, etc.), seals are preferably applied prior to sterilization.

Other aspects of the invention concern methods of cleansing and/or capping medical connectors using a capping and cleansing device according to the invention. Such methods typically involve compressing the resilient inner body of such a device after it has been connected to a medical connector as to allow the cap to spin or rotate in relation to the resilient inner body. Such compression facilitates contact between the device's compressible cleansing matrix and the associated surface(s) of the medical connector. Spinning or rotation of the cap in relation to the resilient inner body, and the associated surface(s) of the medical connector, allow those surfaces to be scrubbed, thereby cleansing them. Preferably, such cleansing methods provide for the disruption of any biofilm present on the surface(s) of the medical connector associated the capping and cleansing device. And in those embodiments where the compressible cleansing matrix contains one or more antimicrobial agents, microbes and pathogens present in such biofilm and/on on such surface(s) are destroyed or rendered nonviable.

Herein, the compressible cleansing matrix of a capping and cleansing device of the invention comprises one or more cleansing agent species dispersed in a substrate. The cleansing matrix substrate can be any substance that can conform, mold, or compress in a manner that enables the effective friction-based cleansing of the site or portion of the medical connector to be cleansed, including the top surface of the site, side surface, and any threads or grooves, if present, and provide the cleansing agent at least at a surface level. Examples of the compressible cleansing matrix include cotton, open or closed cell foam such as polyethylene foam, or other substance that can hold or carry the cleansing agent.

In some embodiments, the cleansing agent species is (are) dispersed in or otherwise combined with the compressible cleansing matrix during the process used to manufacture the capping and cleansing device, while in other embodiments, the device is configured such that the cleansing agent(s) is(are) released for dispersion into the compressible cleansing matrix post-manufacture, but when or prior to the time the matrix is brought into contact with the medical connector to be cleansed. The cleansing agent can be any chemical, substance, or material that cleans the site of bacterial or even viral microorganisms, biofilm, etc., or any carrier that contains such chemical, substance or material. Examples of cleansing agents include isopropyl alcohol, chlorhexidine, chlorhexidine digluconate, povidone-iodine, hydrogen peroxide, soap, and hydrochloric acid, silver ions and salts (e.g., silver acetate, silver lactate, silver nitrate, etc.), etc.

In accordance with the invention, a cleansing agent comprises an active ingredient capable of cleansing a surface of a needleless medical valve. Any active ingredient that can be used effectively to rapidly cleanse a medical fitting or medical line connector (e.g., a needleless medical valve) can be adapted for use in practicing the invention, and are generally classified as antibacterial and/or antifungal agents, antiseptic or antimicrobial agents, wide spectrum disinfectants, and/or parasiticides, as well as combinations of such reagents. Particularly preferred are biocompatible cleansing agents, as the devices of the invention are intended for human and/or veterinary use, including alcohols, antibiotics, oxidizing agents, and metal salts. Representative examples of such active ingredients include bleach, chlorhexidine, ethanol, isopropyl alcohol, hydrogen peroxide, sodium hydroxide, and an iodophor dissolved or otherwise dispersed in a suitable solution, suspension, or emulsion. Other active ingredients having suitable cleansing effects can also be used. These include alcohols (e.g., ethanol, benzyl alcohol, isopropyl alcohol, phenoxyethanol, phenethyl alcohol, etc.); antibiotics (e.g., aminoglycosides, such as amikacin, apramycin, gentamicin, kanamycin, neomycin, netilmicin, paromomycin, rhodostreptomycin, streptomycin, and tobramycin; bacitracin; chloramphenicol; erythromycin; minocycline/rifampin; tetracycline; quinolones such as oxolinic acid, norfloxacin, nalidixic acid, pefloxacin, enoxacin and ciprofloxacin; penicillins such as oxacillin and pipracil; nonoxynol 9; fusidic acid; cephalosporins; etc.), quaternary ammonium chlorides; quaternary ammonium carbonates; benzalkonium chloride; chlorinated phenols; fatty acid monoesters of glycerin and propylene glycol; iodine; iodine containing compounds, such as 3-iodo-2-propynyl butyl carbamate (IPBC); iodophors, such as povidone-iodine (Betadine 100%, which contains providine iodine as the active ingredient); hydantoins, such as dimethylhydantoin and halogenated hydantoins; isothiazolinones; parabens, such as methylparaben, ethylparaben, and propylparaben; chloroxylenol; chlorhexidine and its salts; chlorhexidine/silver-sulfadiazine; chlorhexidine acetate; chlorhexidine gluconate (e.g., Hibiclens); chlorhexidine hydrochloride; chlorhexidine sulfate; benzoic acid and salts thereof; benzalkonium chloride; benzethonium chloride; methylbenzethonium chloride; chlorobutanol; sorbic acid and salts thereof; imidazole antifungals (e.g., miconazole); butocouazole nitrate; mafenide acetate; nitrofurazone; nitromersol; triclocarban; phenylmercuric nitrate or acetate (0.002%); chlorocresol; chlorbutol; clindamycin; CAE (Anjinomoto Co., Inc., containing DL-pyrrolidone carboxylic acid salt of L-cocoyl arginine ethyl ester); cetylpyridinium chloride (CPC) at 0.2%, 0.02%, and 0.002% concentrations; 9.8% isopropyl alcohol; 1% ZnEDTA; mupirocin; and polymyxin (polymyxin b sulfate-bacitracin). Additionally, other useful compounds and compositions include Miconazole, Econazole, Ketoconazole, Oxiconizole, Haloprogin, Clotrimazole, butenafine HCl, Naftifine, Rifampicin, Terbinafine, Ciclopirox, Tolnaftate, Lindane, Lamisil, Fluconazole, Amphotericin B, Ciprofloxecin, Octenidine, Triclosan (2,4,4′-trichloro-2′-hydroxydiphenyl ether), Microban (5-chloro-2phenol (2,4 dichlorophenoxy). Useful metals include silver and its salts, including silver acetate, silver benzoate, silver carbonate, silver citrate, silver iodate, silver iodide, silver lactate, silver laurate, silver nitrate, silver oxide, silver palmitate, silver protein, and silver sulfadiazine. Cleansing reagents are often compositions that comprise the desired active ingredient(s) in admixture with other ingredients, such as carriers and liquid solvents.

The particular active ingredient(s) selected as a cleansing agent for a given application will be compatible with the compressible cleansing matrix and material(s) used to form the cap, resilient inner body, and other components of the particular device. In some embodiments, the cleansing agent is dispersed in the compressible cleansing matrix after the matrix is formed. For example, a cleansing agent can be dispersed by saturating or supersaturating a compressible cleansing matrix during manufacture of the device, preferably before it is sealed. In other embodiments, the cleansing agent can be dispersed during the process used to manufacture the compressible cleansing matrix. As will be appreciated, the materials used to prepare the cleansing agent should be compatible with the constituent or constituents that comprise the compressible cleansing matrix such that the substrate does not appreciably degrade or otherwise suffer loss of structural integrity prior to being used to cleanse a medical connector (e.g., a needleless medical valve). Similarly, the cleansing agent should be biocompatible, such that it will not harm a patient in the event of contact or should some amount of the cleansing agent be admitted into the fluid-carrying portion of a needleless medical valve, as well as with materials used to form needleless medical valves (or other medical connector).

In preferred embodiments, the material used to form the compressible cleansing matrix is any suitable absorbent, compliant, pliable, resilient, fibrous, or porous material, or combination of materials, than can be wetted and/or impregnated with a cleansing agent and which can easily and readily adapt to complex surface contours (e.g., luer threads, concave and convex surfaces, flanges, etc.) likely to be engaged upon contact with, for example, a needleless medical valve to be cleansed. Such materials include those that are synthetic or naturally occurring, and they may be of homogeneous or heterogeneous composition. Preferred synthetic materials include fibrous, foam, and gel compositions, particularly those having directionally oriented natural or synthetic fibers, or combinations thereof. Preferred naturally occurring materials useful as substrates include fibrous naturally occurring materials, including plant-derived materials such as cotton and paper products, as well as animal-based fiber products such as wool. Other preferred natural materials are sponges.

As will be appreciated, in order to achieve the desired cleansing effect, a compressible cleansing matrix, or the component part(s) thereof designed to contact a medical connector such as a needleless medical valve, preferably are made of a material (or combination of materials) that allow the cleansing element to thoroughly cleanse surfaces of medical connectors such as needleless valves or luer access devices, particularly those surfaces that are exposed to air and thus are at risk for contamination with infectious or pathogenic agents, and biofilms containing the same, and are also intended to form part of the fluid flow path for fluids to be introduced into a patient, for example, IV solutions, medications, blood and blood products, etc.

Preferably, the material used to produce the compressible cleansing matrix should be sufficiently compliant to allow the compressible cleansing matrix to deform under the pressures experienced during normal use in order to allow it to conform to the external structures present on the surface(s) of the medical connector to be cleansed. This assures intimate, cleansing contact between the compressible cleansing matrix and at least those exposed surfaces of, for instance, a needleless medical valve designed to come into contact with fluid entering the valve, such as IV fluids. In addition, the compressible cleansing matrix preferably allows for the retention of a liquid cleansing agent, for example, in capillary spaces, in the void volume of foams, sponges, etc. The compressible cleansing matrix may also be engineered such that it includes cleansing agents such as silver ions and/or other suitable materials.

Preferred natural materials from which compressible cleansing matrices can be formed include those derived from cotton and naturally occurring sponges. As those in the art appreciate, processed cotton fibers are composed almost entirely of the natural polymer cellulose. In such fibers, 20-30 layers of cellulose are coiled into a series of spring configurations, which makes the fibers absorbent and gives them a high degree of durability and strength. For example, woven cotton sheets, as are often used in the manufacture of sterile cleansing pads that are then saturated with a 70% isopropyl alcohol (IPA) solution, can be used as substrates for cleansing elements according to the invention. Any suitable configuration may be used. For example, a woven cotton sheet can be cut into numerous similarly sized pieces, each of which can be used as a substrate. In many embodiments, after attachment to the inside surface of a layer of the container (e.g., through the use of an adhesive, double-sided, tape, etc.), the matrix is ready for the addition of a suitable cleansing agent. Alternatively, cotton fibers can be spun onto the inside surface of the cap. Other fibers, be they naturally occurring, synthetic, or combinations of natural and synthetic materials, having similar properties can also readily be adapted for use as compressible cleansing matrices.

Another class of materials for compressible cleansing matrix fabrication is directionally oriented fibrous materials. These include, without limitation, materials comprised of cellulose fibers, glass fibers, and polyester fibers, as well as materials comprised of combinations of two of more of these and/or other materials. Such bonded synthetic fibers use capillary action to precisely absorb, retain, transfer, and/or release liquids or vapor in desired amounts. A broad range of synthetic polymers can be used to form the fibers, and, if desired, they may be treated for functional purposes, for example, to contain a cleansing agent dispersed therein, to provide a vapor barrier or other coating over a portion of the product's surface, etc. The geometric shape of these materials can also be customized for particular applications, thereby permitting easy integration into substrate configurations having the desired device thickness, widths, length, diameter, etc.

Other representative classes of materials suitable for use as compressible cleansing matrices include gel-forming polymers and foams such as agarose, agar, polyacrylamide, and other synthetic porous materials that can be formed into layers, sheets, columns, or other shapes compatible with practicing the invention. Representative gelatinous materials include hydrogels (i.e., cross-linked polymers that absorb and hold water), particularly those made from agarose, (2-hydroxyethyl)methacrylate and its derivatives, and synthetic carbohydrate acrylamides.

Still other classes of materials include porous polymer sponges. Such sponges can be formed from any suitable material, including polyethylene, polypropylene, olytetrafluoroethylene, polyvinylidine difluoride, polynitrile, and polystyrene. Many such porous polymer sponges are commercially available in a wide variety of shapes, pore density and size, etc. Additionally, polymer sponges can be made by polymerizing appropriate monomers according to conventional foam forming techniques. In general, sponges have an open pore structure to allow movement of a solvent such as a liquid cleansing agent. The sponge surface should include open pores to provide entry of liquid cleansing agents (e.g., alcohol, iodine-containing solutions, etc.), and, as with other materials used to form matrices, the particular material chosen is preferably inert, i.e., not reactive with components of the cleansing agent, the body of the capping and cleansing device, or the materials used to produce medical connectors such as needleless medical valves.

Surgical foams are another preferred class of materials that can be used to make compressible cleansing matrices. The materials can be natural or synthetic, as desired. Suitable foams include rubber latex, polyurethane, polyethylene and vinyl foams. Preferably, such foams are made from any suitable biocompatible polymer, for example, polyvinyl alcohol (PVA) or polyurethane. One preferred foam material is Microbisan™, a hydrophilic polyurethane foam that is impregnated with silver ions (Lendell Manufacturing, St. Charles, Mich.). Preferably, such foams are highly absorbent and thus suitable for use with liquid cleansing agents. In other embodiments, the material used to form the foam is well-suited for dispersion of a dry cleansing agent, such as silver ions. Again, it is preferred that foam materials, if used to as a substrate, be inert. Also, they are preferably sufficiently flexible to conform to the variety of different shapes and surface configurations (e.g., double seal fluid access points, luer threads, etc.) encountered in the field given the multitude of medical valve shapes, sizes, and configurations. In this way sufficient contact between the cleansing surface(s) of the capping and cleansing device and the surface(s) of the medical connector to be cleansed can be ensured. Another advantage of some synthetic foams (as well as certain other polymeric materials from which substrates may be formed) is that they can easily be injected in a desired volume into a shell or housing during manufacture, after which they expand to assume the desired substrate size, density, porosity, etc.

Furthermore, compressible cleansing matrices can include chemicals to indicate a functional change therein, for example, by using a color change to signal a change from a wet to a dry state, or, alternatively, that the matrix material has been properly wetted with a liquid cleansing agent dispersed into the substrate by a health care worker just prior to use, as opposed to during manufacture of the device. Thus, depending on the system used, a color change in the matrix could be used to indicate that the cleansing agent in the compressible cleansing matrix has evaporated prior to use and thus the particular cleansing device should not be used, perhaps due to a leak in the capping and cleansing device's storage container. Alternatively, when, for example, a colored liquid cleansing agent is used, the user can visually confirm dispersion of the reagent in the matrix by assessing whether the colored cleansing agent is dispersed throughout the matrix. When colored cleansing agents are used, it is preferred that the material(s) use to make the resilient inner body and/or cap of the capping and cleansing device be clear or translucent, or include one or more clear or translucent windows, in order to allow easy visualization of any color change prior to or during use of the capping and cleansing device.

Capping and cleansing devices of the invention and their constituent parts (e.g., the resilient inner body, cap, compressible cleansing matrix, sealing ring, seal, etc.) can be made from any suitable material(s) and assembled using any suitable process.

Preferably, the outer surface of a capping and cleansing device's cap intended for grasping by a user has a non-slip surface, i.e., one having a high coefficient of friction so that when the cap portion of a capping and cleansing device is held in a user's fingers and positioned to cleanse a medical connector, for example, by applying pressure to the cap along its central axis so as to compress the resilient inner body and thereby disengage the engaging elements locking the cap and inner body together, the cap can be rotated in relation to the resilient inner body and medical connector with minimal or no slippage between the cap and the user's fingers (gloved or ungloved). Examples of such non-slip (or high friction) surfaces include those having ridges, valleys, dimples, bumps, or other features designed to enhance friction, as well as combinations of two or more of such features. Such features can be introduced into a cap's outer surface(s) as part of the manufacturing process. Alternatively, a non-slip coating can be applied to one or more of the outer surfaces of the cap.

In general, the capping and cleansing devices of the invention are provided to users in a sealed, sterile manner. If desired, labeling information, logos, artwork, manufacturing, and/or regulatory data (e.g., lot number, expiration or “use by” dates, etc.) may also be printed or otherwise applied to individual capping and cleansing devices. In addition, information such as a bar code (to allow use of the device to tracked, for example) may also be included on individual capping and cleansing devices.

As will be appreciated, cleansing devices may be packaged individually or in groups of two or more units as kits, which can further include instructions for use of the capping and cleansing device(s), as well as other information, logos, artwork, manufacturing, and/or regulatory data.

In preferred embodiments, packaged capping and cleansing devices are sterilized using a suitable process, such as irradiation. In a particularly preferred practice, the capping and cleansing device s of the invention are sterilized as part of the manufacturing process. Here, “sterilization” refers to any process that effectively kills or eliminates transmissible agents, e.g., bacteria, viruses, fungi, prions, spores, etc. that may be present in any component of a device according to the invention. In preferred embodiments, sterilization can be achieved by heating, chemical treatment, irradiation, and other processes. Indeed, any sterilization process compatible with the materials used to make the capping and cleansing device can be employed. A particularly preferred sterilization process is an irradiation process. Such processes include irradiation with x-rays, gamma rays, or subatomic particles (e.g., an electron beam). In general, when a sterilization process is used in the context of the invention, the process is employed on a cleansing article after it has been sealed and/or packaged. Chemical sterilization processes can also be used, for example, sterilization using ethylene oxide (EtO).

The invention also concerns methods of using the instant single-use capping and cleansing devices of the invention. Such methods include using the articles to cleanse and, if desired, cap medical connectors such as needleless medical valves, luer access devices, and the like. To perform such methods, the portion of a medical connector to be cleansed is threaded into the central bore of the resilient inner body of a capping and cleansing device, typically after the user (e.g., a nurse) removes a seal that spans the opening in the cap. Such insertion brings the site of the medical connector into contact with (i.e., brought into cleansing association with) the compressible cleansing matrix portion(s) of the device. In preferred practice, once the compressible cleansing matrix is in contact with the surface(s) of the medical connector to be cleansed, the cap of the capping and cleansing device is pushed downward along its central axis to disengage the engaging elements in the cap and resilient inner body to allow cap rotation in relation to the resilient inner body and medical connector previously releasably connected to capping and cleansing device. Such contact and cleansing action can be for any desired period, with periods of about one second to about ten to twenty seconds being particularly preferred.

After cleansing, the medical connector can be removed from the capping and cleansing device, after which the capping and cleansing device may be discarded. Alternatively, after cleansing, the capping and cleansing device can be left attached to the medical connector, capping a portion thereof until such time as access to the medical connector is desired, capping it and protecting it from contamination. At that time, the capping and cleansing device can be removed and discarded. If desired, just prior to removal, a cleansing process can be repeated.

After removal of a capping and cleansing device from a cleansed medical connector, a fluid-containing medical reservoir (e.g., a syringe containing a medication, an IV bag, etc.) may be immediately connected to the cleansed medical connector. In preferred embodiments where the cleansing agent is a solution, the surface(s) of the medical connector is preferably allowed to dry (or is(are) dried, for example, by wiping with a sterile, absorbent cloth or wipe, which cloth or wipe may be dry or wetted with a volatile, compatible solution such as 70-100% alcohol) prior to connecting the medical connector to a fluid reservoir. In preferred practice, such cleansing methods result in at least a 2-fold, 5-fold, or 10-fold or more reduction in microorganism contamination on the accessible surface(s) that have been cleansed. Even more preferably, the level of reduction may exceed a 100-fold, a 10³-fold, a 10⁴-fold, a 10⁵-fold, a 10⁶-fold, or 10⁷-fold reduction in microorganism contamination on the accessible fitting surface.

In addition to methods for cleansing accessible surfaces of luer access devices and the like, the articles of the invention provide methods of reducing infection risk in a patient connected to devices, such as a peripheral IV line, a central IV line, or a peripherally inserted central catheter, configured for delivering fluids directly into the patient's blood stream. The risk reduction afforded by the articles of the invention may vary depending upon many factors, such as patient age and condition, the condition being treated, the location where medical services are being delivered, patient density, the level of contaminating microorganisms in the environment, the quality of air handling equipment in the medical facility, the degree of training of medical personnel charged with cleansing the access device, the method(s) used to periodically cleanse the medical fitting, intervals between cleansing procedures, the particular configuration of the capping and cleansing device, the particular configuration of the medical connector, whether the capping and cleansing device is left on the cleansed site of the medical connector in order to provide capping, etc. Risk reduction can be established using any suitable method, for example, by assessing HAI frequency in the presence and absence of using cleansing devices according to the invention. Reductions of HAI infection risk of 1-100% or more, including up to 1000% or more, are envisioned through use of capping and cleansing devices according to the invention. As will be appreciated, reductions in infection risk (e.g., HAI risk) will translate to improved patient outcomes (through reduced morbidity and mortality) and reduced expenditure on treating HAI's.

### Representative Embodiments

To further illustrate and describe certain preferred, representative embodiments of the invention, the reader is directed to the appended drawings, FIGS. 1-6, which illustrate various particularly preferred embodiments of the capping and cleansing devices of invention. A description of these preferred, representative embodiments follows.

FIG. 1 shows several drawings ((a)-(g)) of a representative capping and cleansing device of the invention (10), its constituent parts (views (b)-(g)), and the device associated with a medical connector (view (a)). The constituent parts include a cap having a cap portion (11) adapted to receive and retain the compressible cleansing matrix (80) and a resilient inner body (30) associated with the cap portion (11) and adapted to engage one or more complementary features of the cap portion so as to prevent the cap portion and resilient inner body from moving independently of each other under certain conditions while under other conditions allowing the cap portion (11) and to move independently of each other. For example, after attaching the device (10) to a medical connector (100) (producing a capped medical connector (200)), a user an compress the resilient inner body (30) of the device (10) by applying pressure to compress the cap (10) against the medical connector (100) so as to allow the cap (10) to be rotated in relation to the resilient inner body (30) and the medical connector (100).

Such action brings the compressible cleansing matrix (80) and surface(s) of the medical connector (100) desired to be cleaned, for example, the valve surface of a needleless medical valve, into contact, and rotation of the cap (10) in relation to the medical connector (100) creates friction that can disrupt, for example, biofilm that may be present on the needleless medical valve's valve surface, which surface can be in the fluid path of fluids moving through the medical valve.

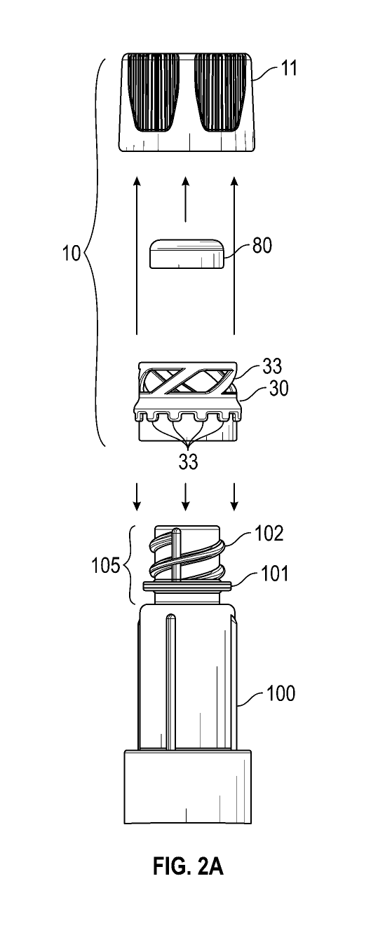

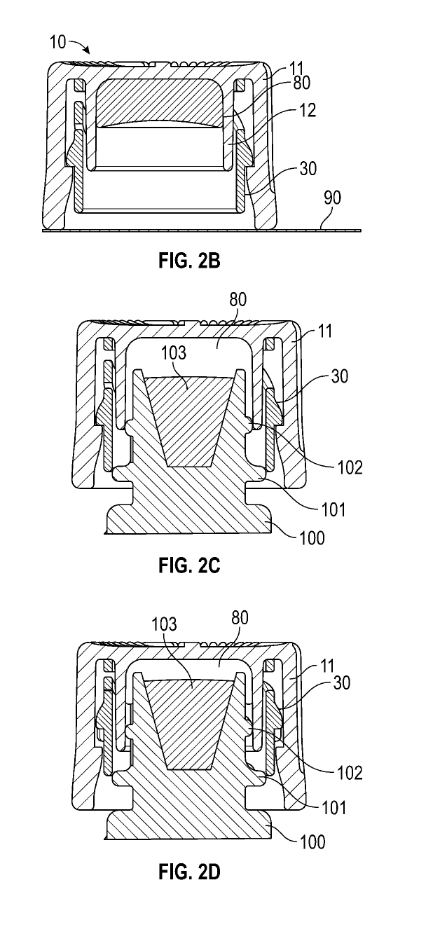

FIG. 2 (a) is an exploded view of a representative capping and cleansing device of the invention (10) and a medical connector (100). Visible are the cap (10), including its cap portion (11) into which the compressible cleansing matrix (80) and resilient inner body (30) are positioned, and a luer-based medical connector (100) that is a needleless medical valve, the male end (105) of which has a collar (101) and threads (102) for connecting the valve to a female threaded portion of a complementary luer fitting of another medical connector (not shown). View (b) is a cross-section side view of the cap (10) shown in view (a) while it was still sealed with a seal (90). As shown, the cap portion (11), compressible cleansing matrix (80), and resilient inner body (30) are operably assembled. The compressible cleansing matrix (80) is preferably positioned in a matrix well (12) formed into and protruding from the inner surface of the cap portion (11). The height of the matrix well (12) should allow retention of the compressible cleansing matrix (80), and in some embodiments it can be sized to act as a stop that can bear against the collar of a medical connector (100) to which the cap is attached when the cap is compressed by a user and rotated to cleanse desired surfaces of the medical connector (100).

Views (c) and (d) of FIG. 2 show the cap (10) threaded onto the medical valve (100) in capping and cleansing configurations (views (c) and (d), respectively). As shown in these views, the compressible cleansing matrix (80) of the cap (10) bears against the valve surface of the valve stem portion (103) of the medical valve (100). In the capping view, view (c), the cap (and compressible cleansing matrix (80)) is not compressed. The diameter of the matrix well (12) allows the cap to slide over the threads (102) of the medical valve (100) when the cap is pushed toward the medical valve (100) by user wishing to cleanse the surface of the valve stem portion (103). View (d) shows the cap (10) compressed against the medical valve (100). User-induced compression results in the cap portion (11) moving closer to the body of the medical valve (100) by virtue of compression of the resilient inner body (30) and compressible cleansing matrix (80). This motion also results in disengagement of the complementary mechanical retaining elements of the cap portion (11) and resilient inner body (30), thus allowing the user to rotate cap portion (11) and the compressible cleansing matrix (80) of the cap in relation to the valve surface, thereby allowing cleansing of that surface.

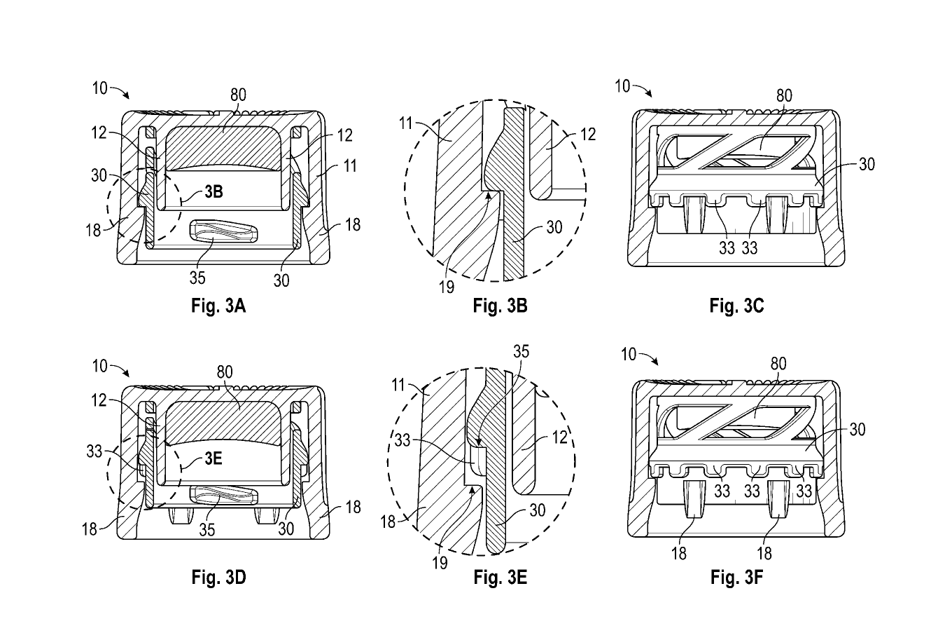

FIG. 3 shows six different views of a representative capping and cleansing device of the invention. Views (a)-(c) show the device (10) in a static, non-compressed, non-rotable position, where the cap portion (11) and resilient inner body (30) are engaged such that the cap (10), and hence the compressible cleansing matrix (80) associated therewith, cannot rotate in relation to the device's resilient inner body (30). Views (d)-(f) show the same representative device (10) with the cap portion (11) and resilient inner body (30) in movable relation such that the cap portion ((11), and the compressible cleansing matrix (80) associated therewith) can be rotated in relation to the device's resilient inner body (30). The inner surface of the resilient inner body (30) includes one or more (preferably two) tabs (35) to engage the threads of a medical connector (100). In the embodiments shown in the Figures, the outer surface of the resilient inner body (30) includes a plurality of teeth (33) or other structures spaced about the resilient inner body's outer circumference designed to engage complementary spaced structures (e.g., ribs (18)) spaced on the inner surface of the cap portion (11). When the cap (10) is uncompressed, the teeth (33) engage the ribs (18) and effectively lock the cap portion (11) and resilient inner body (30) together so that they rotate together. This enables the cap (10) to be threaded, for example, onto a complementary luer fitting of a medical connector using the tabs (35) on the inner surface of the resilient inner body (30) in order to provide a capping function (to remove the cap from the medical connector, the process is reversed). To provide cleansing action, once secured to the medical connector, the cap (10) can then be compressed by a user, which pushes the cap portion (11) toward the medical connector's fitting and compresses the compressible cleansing matrix (80) against the surface(s) of the connector (100) to be cleansed.

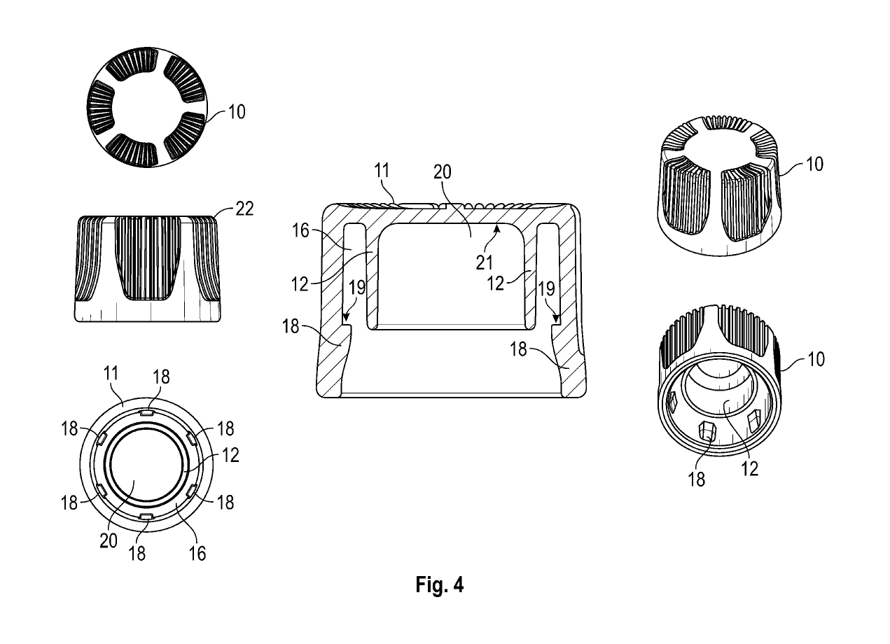

FIG. 4 shows views of the cap portion (11) of a representative capping and cleansing device (10) of the invention. View (a) shows a top view of the cap portion (11). Also visible on portions of the outer surface of the cap portion (11) are ridges and valleys that provide for enhanced friction, allowing a user to better grip or grasp the cap (10). View (b) shows a side view of the cap portion (11). View (c) shows a bottom view of the cap portion (11). Visible in this embodiment are six ribs (or protrusions) (18) evenly spaced (here, about 60 deg. on center) about the circumference of cap portion's inner surface. The ribs (18) are positioned and sized to engage complementary features on the outer surface of the resilient inner body (30)(not shown). The wall forming the matrix well (12) is also visible in this view. View (d) shows a cross-sectional view of the cap portion (11). The well (20) formed by the matrix well wall (12) that extends from the inner surface of the upper portion of the cap portion (11) is also represented, and is adapted to receive and retain the compressible cleansing matrix (80). In preferred embodiments, an adhesive (not shown) or other bonding agent is used to adhere the compressible cleansing matrix (80) inside the well (20). The well (20) is spaced from the outer wall of the cap portion (11). The resulting space is sized and adapted for insertion of the resilient inner body (30), about which the cap portion (11) can be rotated when the complementary retaining elements (e.g., ribs (18) and teeth (33)) of the cap portion (11). In the representative embodiment of the inventive capping and cleansing device (10) depicted in the Figures, the upper surface (19) of the retaining elements (18) present in the cap portion (11) are designed to engage the lower surface of the locking grooves (34) between the teeth (33).

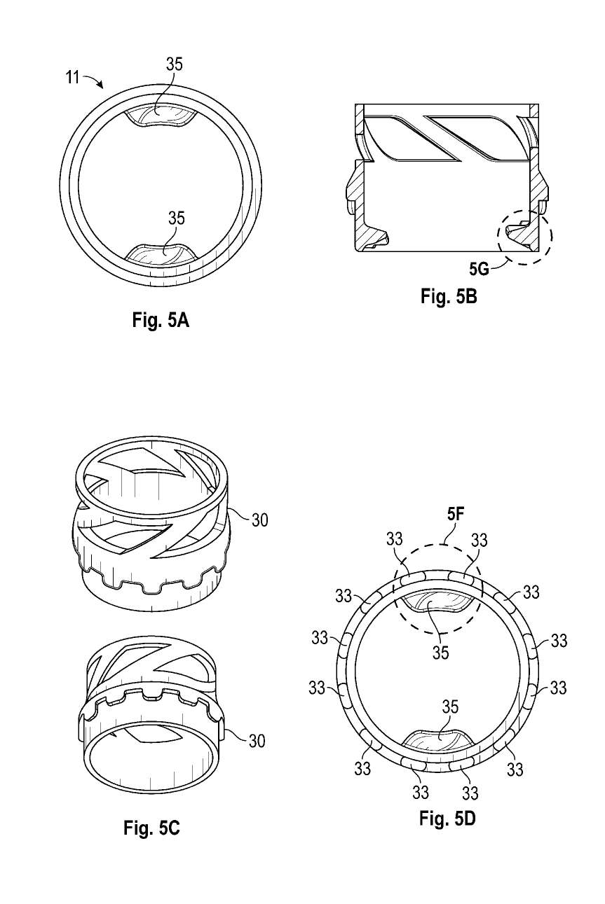

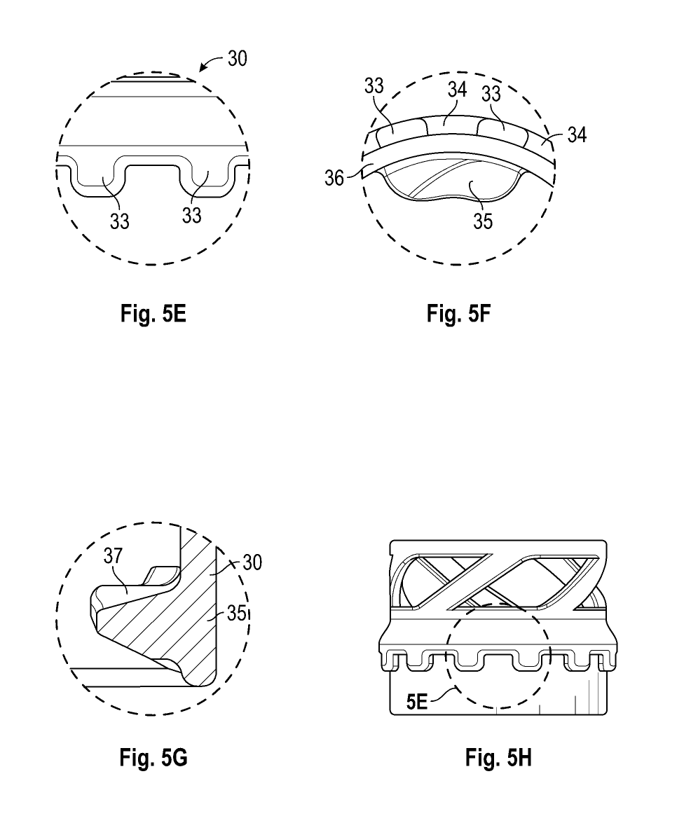

FIG. 5 shows seven different views ((a)-(g)) of the resilient inner body (30) of a representative capping and cleansing device of the invention (10). Representative measurements of this particular embodiment are shown on several of the views. Depicted in this embodiment are two thread tabs (35) disposed on the inner surface of the wall (36) of resilient inner body (30), 12 spaced teeth (33) to engage 12 complementary retaining elements (e.g., ribs (18)) on the inner surface of the cap portion (11). The resilient inner body (30) is adapted for compression by a user upon application of a suitable force, and rebound upon relieving of such pressure.

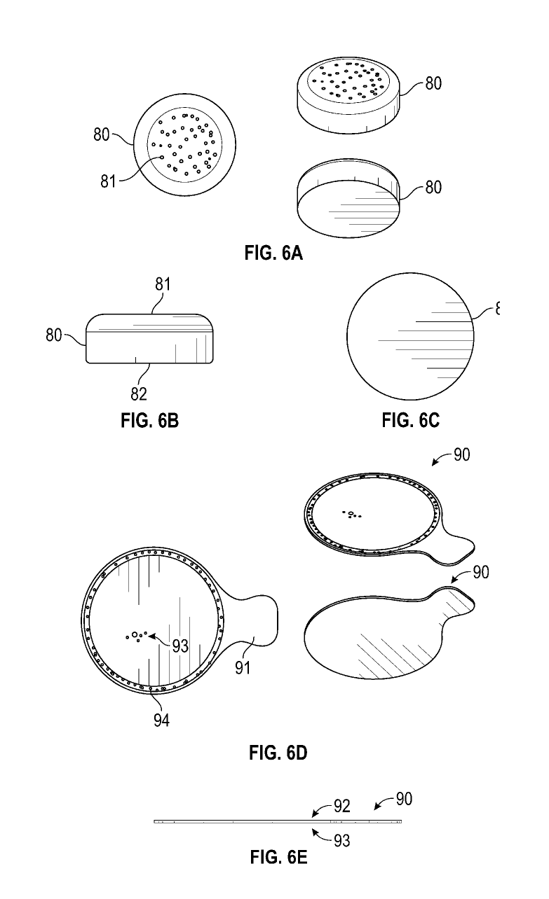

FIG. 6 shows five different views ((a)-(e)), three of which show a compressible cleansing matrix portion (80) of a representative capping and cleansing device of the invention (10). Views (a)-(c) show top, side, and bottom views of this particular compressible cleansing matrix (80). Preferably, the compressible cleansing matrix (80) is adhered using an adhesive to the surface of matrix well (20) of the cap portion (11). Views (d) and (e) of FIG. 6 show bottom and side views of a seal portion (90) of a representative capping and cleansing device of the invention. The seal (90) is typically sized to seal or cover the opening that allows access to the interior of the cap (10). Preferably, the seal contains one or more removal tabs (91) configured to allow grasping by a user such that the seal can be removed just prior to the device being used to cap and/or cleanse a medical connector (100). Preferably, the seal is adhered to the cap (10) using a suitable adhesive (94) applied to the inside surface (92) of the seal (90). The seal's outside surface (93) often will contain alphanumeric characters, bar code information, or the like.

Unless the context clearly requires otherwise, throughout the description above and the appended claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number, respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list, and any combination of the items in the list.

The foregoing description, for purpose of explanation, has been described with reference to specific embodiments. However, the illustrative discussions above are not intended to be exhaustive or to limit the invention to the precise forms disclosed. Many modifications and variations are possible in view of the above descriptions. The embodiments were chosen and described in order to best explain the principles of the invention and its practical applications to thereby enable others skilled in the art to best utilize the invention and various embodiments with various modifications as are suited to the particular use contemplated. As such, the invention extends to all functionally equivalent structures, methods, and uses, such as are within the scope of the appended claims, and it is intended that the invention be limited only to the extent required by the applicable rules of law.

What is claimed is:

1. A capping and cleansing device for a medical connector having a threaded portion, the capping and cleansing device comprising:
(a) a resilient inner body comprising a first opening to a central bore and a second opening opposite the first opening, wherein proximate to the first opening and disposed on an inner surface of the central bore is(are) one or more thread-engaging tabs or threads configured to engage a threaded portion of a medical connector so as to retain, and, if and when desired, release the medical connector; a cap-engaging region configured to allow to mechanical engagement of and disengagement from a cap; and a compressible region to allow compression of the resilient inner body;
(b) a cap comprising an outer cavity formed by an outer wall and a top wall, a concentric central matrix well spaced from the outer wall to form a resilient member housing between the outer wall and central matrix well and into which the compressible region of the resilient inner body is positioned, and one or more locking structures configured to releasably engage the cap-engaging region of the resilient inner body, wherein when the locking structure(s) of the cap are engaged with the cap-engaging region of the resilient inner body the cap and the resilient inner body can be rotated in unison and wherein when the locking structure(s) of the cap are disengaged from the cap-engaging region of the resilient inner body the cap can be rotated in relation to the resilient inner body; and
(c) a compressible cleansing matrix that comprises a cleansing agent, wherein the matrix is secured to an inner surface of the cap top and is configured to contact and cleanse one or more surfaces of a medical connector.

2. A capping and cleansing device according to claim 1, wherein the compressible cleansing matrix includes a first matrix portion secured to the inner surface of a top wall of the cap and a second cleansing matrix portion secured to an inner surface of a wall forming the central matrix well.

3. A capping and cleansing device according to claim 1 that further comprises a removable seal attached to the cap.

4. A capping and cleansing device according to claim 1 that further comprises a sealing ring positioned on an inner surface of the central bore of the resilient inner body proximate to the second opening, wherein the sealing ring is configured to engage a surface of a medical connector upon its insertion into the capping and cleansing device.

5. A capping and cleansing device according to claim 1, wherein the cleansing agent comprises isopropyl alcohol.

6. A capping and cleansing device according to claim 1, wherein the cap comprises an outer surface having a plurality of vertical ridges.

7. A method of cleansing a medical connector, optionally a luer access device, optionally a needleless medical valve, comprising:
(a) connecting a medical connector with a capping and cleansing device according to claim 1 such that the one or more surfaces of the medical connector engage and at least partially compress the compressible cleansing matrix; and
(b) rendering the cap rotable in relation to the resilient inner body by compressing the resilient inner body so as to disengage the locking structure(s) of the cap from the cap-engaging region of the resilient inner body and rotating the cap in relation to the medical connector, thereby cleansing the surface(s) of the medical connector contacted by the compressible cleansing matrix.

8. A method according to claim 7 that further comprises leaving the medical connector connected to the capping and cleansing device after cleansing, thereby capping the medical connector.
